(19) United States
(12) Patent Application Publication
(10) Pub. No.: US 2023/0082919 A1
(43) Pub. Date: Mar. 16, 2023

(54) CANNULA FIXATION DEVICE

(71) Applicant: Inspira-Technologies OXY B.H.N. LTD., Ra'anana (IL)

(72) Inventors: Shlomo Schwartz, Kfar-Shaba (IL); Adi Rizansky NIR, Herzliya (IL)

(21) Appl. No.: 17/931,134

(22) Filed: Sep. 11, 2022

(30) Foreign Application Priority Data
Sep. 10, 2021 (IL) 286271

(51) Int. Cl. A61M 25/02

(52) U.S. Cl. CPC A61M 25/02; A61M 2025/0206; A61M 2025/028

(57) ABSTRACT

This invention is directed to a cannula fixation device comprising at least: an open frame configured to be mounted on a patient's neck, said frame having at least one opening for allowing approach to at least one neck artery and/or vein; at least one cannula holder configured to allow securing of a cannula to said frame; and at least one fastening element for closing the open frame onto the patient's neck and adjusting it to the neck's dimensions, wherein the cannula fixation device is configured to secure at least one cannula inserted into the neck artery and/or vein of said patient and preserve the cannula position within the artery and/or vein in any body positioning, while said patient is lying down, standing, or sitting.

### FIELD OF THE INVENTION

The present invention generally relates to fixation and securing of medical cannula to a patient. More specifically, the invention relates to a novel device for retaining one or more cannulas inserted via a patient's neck in any positioning of the patient, in the hospital and during transporting the patient and, while the patient is awake or sedated.

### BACKGROUND OF THE INVENTION

Many medical procedures require insertion of medical cannulas, tubes, catheters, and lines into the body near the patients' head and neck area. In some cases, the inserted parts require securing/fixation for avoiding undesired movements and accidental removal of the device. For example, a cannula being inserted into the internal or external jugular vein requires sufficient fixation to prevent undesired movement or unintentionally pulling during treatment. Presently, securing of such cannulas is performed by using designated adhesive dressings. However, the efficiency of the adhesive dressings is insufficient, and the adhesive dressings may come loose when high flow capacity through the cannula is required and/or the weight of the apparatus is too heavy to be supported by the adhesive dressings. In addition, the adhesive dressings are uncomfortable and irritating, and may cause a patient to attempt to remove the adhesive dressings so as to get a relief. This is especially the case for treatments such as extracorporeal oxygenation, which may take place over the course of many hours and even several days. Some attempts were made in the art to solve this problem. The following references disclose various devices designed for such purpose and may be considered as related to the field of this invention: U.S. Pat. Nos. 9,789,287; 10,695,473; 5,336,195; CN Utility Model No. 213609268; and U.S. Pat. No. 10,137,278. Some of the devices disclosed in the above references are either cumbersome and very uncomfortable to the user and to the medical team, as it is difficult to mount them on the patient and further to remove them. Some of them are flexible and their efficiency for this purpose is limited. Additionally, the approach to the cannulated area is limited or partially limited and make it difficult to access, and even limits the medical team's ability to perform further medical procedures that may be required during or after cannulation. In addition, the unique structure of the novel device provided herein allows for bi-lateral cannulation of a patient.

Additional need that the novel device provided herein may resolve, relates to transportation of ECMO (Extracorporeal membrane oxygenation) patients to a referral medical center (specializing with ECMO). The transfer occurs in many cases when the patient is connected to ECMO. Majority of hypoxemic patients which ultimately receive ECMO treatment are hospitalized in hospitals without ECMO centers and transported to ECMO centers. The transportation of patients to ECMO center requires reliable fixation of the Jugular cannula as it is a matter of life saving. Additionally, transportation of cannulated patients should not be limited to ECMO patients, and the novel cannula fixation device of this invention may be used for other cases requiring transportation of cannulated patients and may be applied to any scenario involving evacuation of casualties from the area of an event to hospitals.

The novel device provided herein is further directed to support the safe placing of a patient in the prone position (proning) while receiving ECMO treatment. Prone positioning improves gas exchange by ameliorating the ventral-dorsal transpulmonary pressure difference, reducing dorsal lung compression, and improving lung perfusion. Clinical evidence demonstrated the significant advantages of prone positioning, contributing to oxygenation improvement as well as a significant decrease of mortality in mechanically ventilated, acute respiratory failure patients (see for example Guérin, Claude, et al. “Prone positioning in severe acute respiratory distress syndrome.” New England Journal of Medicine 368.23 (2013): 2159-2168).

In recent years, few studies have explored the benefits of prone positioning in respiratory failure patients treated with ECMO (see for example Giani, Marco, et al. “Prone positioning during venovenous extracorporeal membrane oxygenation in acute respiratory distress syndrome. A multicenter cohort study and propensity-matched analysis.” Annals of the American Thoracic Society 18.3 (2021): 495-501.) As, with mechanically ventilated patients not receiving ECMO treatment, prone position in patients treated with ECMO showed significant benefits in oxygenation and overall survival.

Unlike the proning of patients connected to mechanical ventilation only, the proning of patients treated with ECMO is subjected to complication resulting from the ECMO cannula movement or dislodgment. The ECMO patient is connected to a large-diameter cannulas permitting ample blood flow to and from his/her body. An undesirable movement or decannulation of the cannula during proning process, or whenever the patient is turned back from a prone to a supine position, may have a lethal outcome. For that reason, proning of ECMO is not pursued in many hospitals providing ECMO treatments, albeit its evidence based clinical benefits. The novel device provided herein is configured to allow safe proning of ECMO patients.

### SUMMARY OF THE INVENTION

This invention is directed to a novel device for retaining cannulas inserted via a patient's neck by securing the cannula to the device of the invention that is mounted onto the patient during the medical procedure as will be described in detail hereinbelow. The novel device provided herein may be used hospitals and medical centers and outside during providing first aid, and it may be used in all patient's body positioning including during proning.

The term “cannula/s” as used herein also refers to tubes, catheters, and lines that may be placed in blood vessels for performing various medical procedures such as but not limited to administer fluids, blood, medications, and nutrition to the patient, provide the patient a medical treatment that involve utilization of medical device such as but not limited to, ECMO, dialysis and else. For simplicity of explanation, reference to all of the above (cannula, tubes, catheters, and lines) is made by refereeing herein to a single term: “cannula/s”, which may practically and interchangeably refer to either one of them. Furthermore, in practice, the cannula inserted into the patient's artery/vein may be extended by connecting it to a tube that may be further connected to a medical device/system such as ECMO, cardiopulmonary bypass machine, dialysis machine. plasmapheresis machine, and else.

The novel device provided herein may be made of a rigid or semi-rigid material, it may be fully or partially disposable, and it can easily be removed from the patient's body while keeping the cannula in place (in contrast to prior art devices in which it is hard to do, if possible, at all). Furthermore, the novel device provided herein is configured to fixate the tubes and not to fixates the cannula tip, and it may be bi-directional to thereby allow attachment of cannulas from both sides of the patient's neck and to enable clear approach to the cannula entry point for performing various treatments required such as cleaning the area, placing a bandage and the like. The novel cannula fixation device provided herein may be applied to patients and may be used in any body positioning of the patient including proning, it may be used in hospital, medical centers and outside during transporting the patient from one medical center to another, at the hospital from one spot to another, and from the event site to a medical center. It may be used while the patient is awake or sedated.

Thus, in one main aspect, this invention is directed to a cannula fixation device comprising at least: a) an open frame configured to be mounted on a patient's neck, said frame is encircling the patient's neck and having at least one opening at the cannula entry point for allowing approach to at least one neck artery and/or vein; b) at least one cannula holder configured to allow securing of a cannula to said frame; and c) at least one fastening element for closing the open frame onto the patient's neck and adjusting it to the neck's dimensions; wherein the cannula fixation device is configured to secure at least one cannula inserted into the neck artery and/or vein of said patient and preserve the cannula position within the artery and/or vein in any body positioning, while said patient is lying down, standing, or sitting.

The frame may be made of a rigid or semi-rigid material. The frame may be composed of two or more frame sections sequentially connected to each other. In some embodiment, when the frame is composed of two or more frame sections, the cannula fixation device may further comprise connectors for connecting said frame sections.

In accordance with some optional embodiments, the frame comprises a left opening and a right opening so as to enable simultaneous approach to the left and right neck arteries and veins.

Yet, in some further embodiments, the cannula fixation device may further comprise an inner soft layer attached to the frame and covers the frame either fully or partially. Optionally, the inner layer may extend beyond the frame in some areas of the frame, such as the neck area.

The at least one cannula holder may be part of the frame or an independent connecting element attached thereto. In accordance with some optional embodiments, the at least one cannula holder is a curved section of the frame creating a niche.

In a specific embodiment, the niche may have two holes at the right and left sides of the niche that allows to secure the cannula to the frame. Optionally but not necessarily, the at least one cannula holder may be either one of clips, straps, adhesive tapes, at least two subsequent mechanical projections, clasp, cable tie, plastic or nylon restraint.

Optionally, the frame further comprises at least two projections configured to hold the ascending portion the cannula and fixate it to the frame. The frame may be composed of different heights at different areas around the neck and various designs may be used for different patients. The at least one fastening device used may be selected from the group consisting of Velcro strap, elastic modular strap, clips, straps, adhesive tape, clasp, or else.

The cannula fixation device described herein may be used when a patient is intubated for either one of the following: extracorporeal blood processing, cardio-vascular therapeutic or diagnostic procedure, hemodialysis, plasmapheresis, transvenous cardiac pacing, invasive hemodynamic monitoring, and heart-lung machine. The cannula fixation device of this invention may also be used when the patient is positioned in a proning position.

Optionally, the secured cannula may further be connected to a tube, in a manner that the tube is functionally connected to the frame by the cannula holders. The tube may be supported by a rigid assembly mounted on the portion of the tube that is attached to the frame so as to prevent kink/collapse of said tube. The rigid assembly may be for example, a metal/plastic coil, rigid rings, u shapes, hooks and the like.

This invention is also directed in one main aspect to a cannula fixation device for securing at least one cannula inserted into the neck artery and/or vein of a patient and preserve the cannula position within the artery and/or vein in any body positioning, said cannula fixation device comprising at least: a) an open frame comprising one or more frame segments and configured to be mounted on a patient's neck, said frame having at least one opening in dimensions allowing to approach at least one neck artery and/or vein at the cannula entry point for performing a medical procedure; b) at least one cannula holder configured to allow securing and/or fixating of a cannula inserted into the patient's body to said frame; and c) at least one fastening element for closing the open frame onto the patient's to encircle the neck and adjusting to the neck's dimensions.

Optional embodiments of this invention will be detailed described with reference to the drawing hereinbelow.

### BRIEF DESCRIPTION OF THE DRAWINGS

FIG. 1A is a schematic isometric illustration of a cannula fixation device in a close ready to use form, according to optional embodiments of the invention.

FIG. 1B is a schematic back view illustration of the cannula fixation device of FIG. 1A.

FIG. 1C is a schematic isometric illustration of the cannula fixation device of FIG. 1A in an open form ready to be mounted on a patient.

FIG. 1D is a schematic illustration of the cannula fixation device of FIG. 1A mounted on a patient.

FIG. 1E is a schematic front view illustration of the cannula fixation device of FIG. 1A illustrating neck blood vessels shown through the openings of the device, in accordance with optional embodiments of the invention.

FIG. 2 is a schematic illustration of a cannula fixation device according to one another embodiment of the invention.

FIG. 3 is a schematic illustration of a cannula fixation device according to one another embodiment of the invention.

FIG. 4 is a schematic illustration of a cannula fixation device according to one another embodiment of the invention.

FIG. 5 is a schematic partial close-up view demonstrating the two layers of the cannula fixation device.

FIG. 6 is a close-up view of a tube with a supporting accessory for preventing its collapse according to some embodiments of the invention.

### DETAILED DESCRIPTION OF EMBODIMENTS OF THE INVENTION

The present invention is aimed to provide a novel fixation device for securing a medical cannula within an artery and/or vein of a patient cannulated at the head and/or neck area, for preventing undesired displacement, intentional or accidental pulling, or undesired movement of the cannula. In addition, the novel device allows to safely reposition the patient and perform, for example, proning during ECMO. The novel device provided herein when compared to prior art devices, is friendly to the patient and may be used for conscious patient that are mobile as well as for anesthetized patients, it allows convenient approach to the treated area, it is simple to mount on the treated area and to remove after use, and it may be partially or fully disposable.

In the following detailed description, reference is made to several optional, none limiting embodiments of the invention, examples of which are illustrated in the accompanying figures. The figures depict some optional embodiments of the invention for purposes of illustration only and should not be construed as limiting the scope of this invention. One skilled in the art will readily recognize from the following description that the described embodiments may be combined, alternative embodiments may be utilized, and other changes may be made without departing from the spirit or scope of the present invention as described herein. Reference is now made to the drawings:

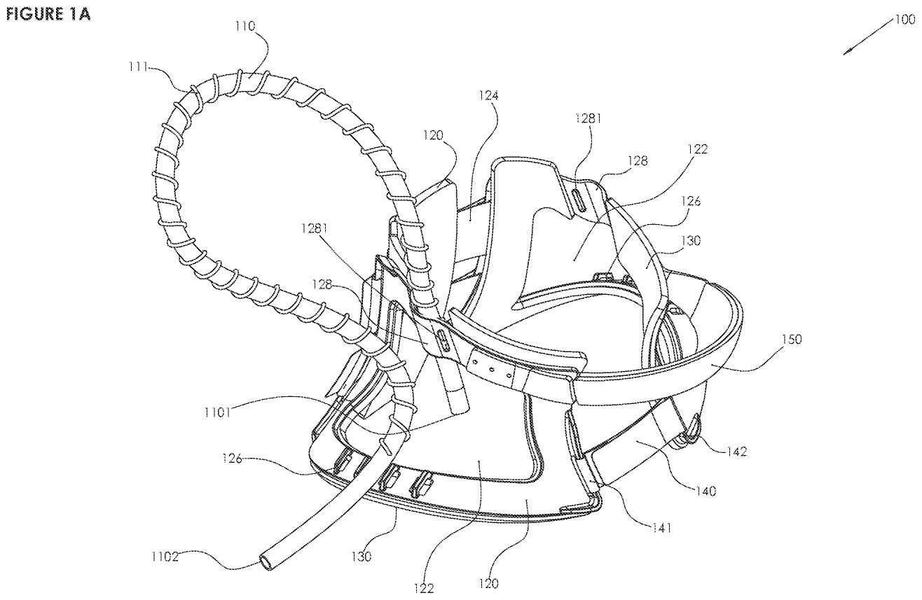

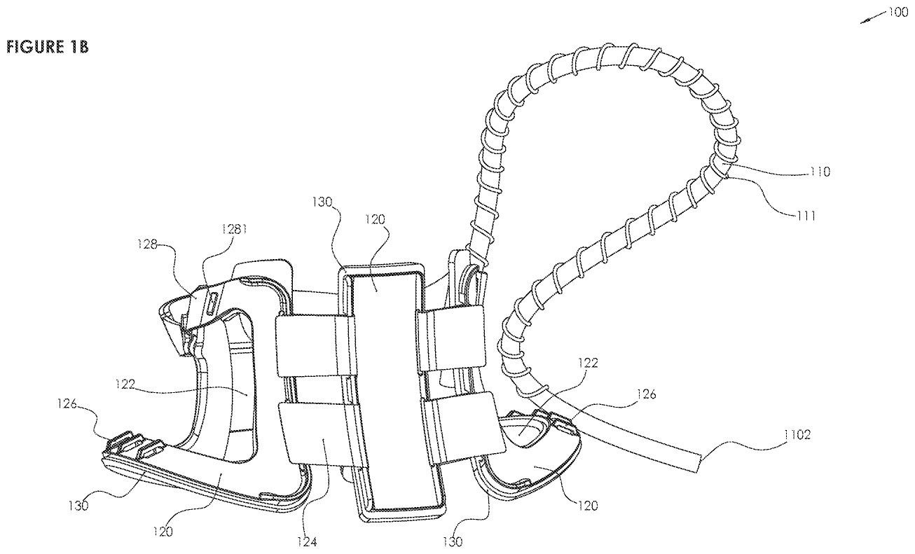

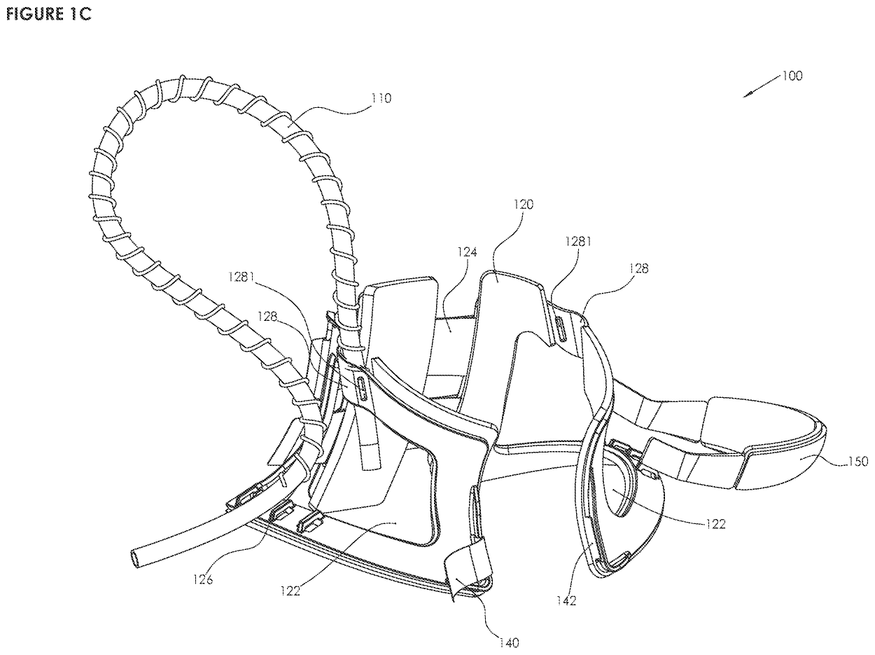

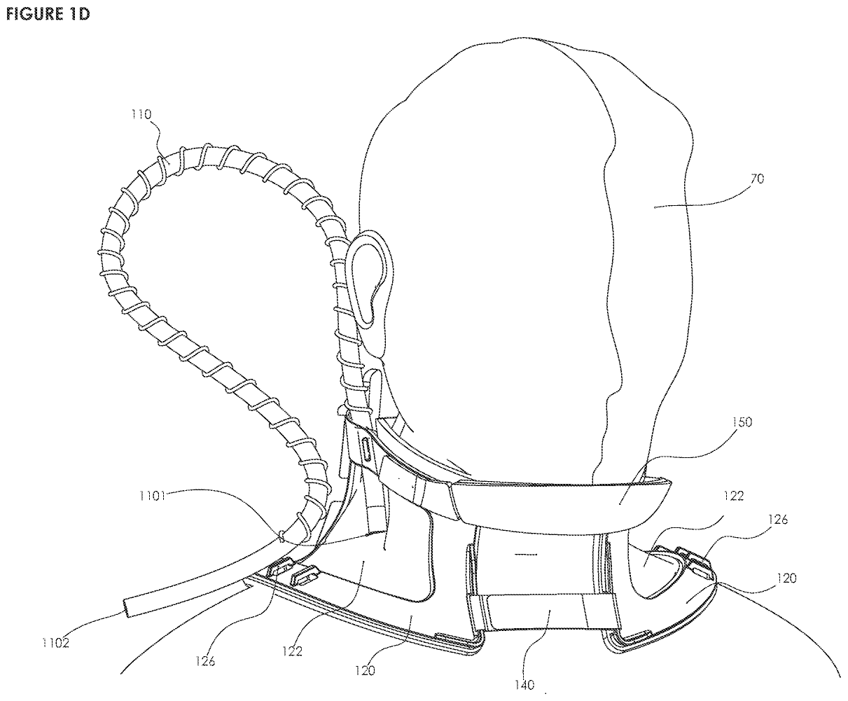

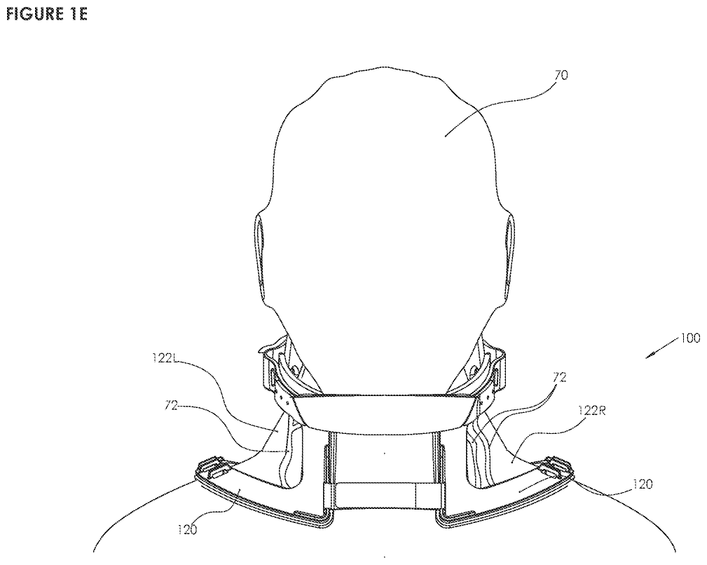

FIGS. 1A-1B are schematic isometric view and back view illustrations respectively of a cannula fixation device 100 in a close ready to use form in accordance with one optional embodiment of the invention.

Device 100 is configured to retain cannula 110. In accordance with some exemplary embodiments, cannula 110 is a cannulation tube carrying blood between a jugular vein and an extracorporeal oxygenation system (not shown). The extracorporeal oxygenation system may be, for example, an ECMO system designed to replace independent breathing, and/or a system designed to supplement independent breathing. It should be clarified that the description herein refers to cannula 110 for simplicity of explanation, however other alternatives to cannula 110 are also within the scope of this invention and the cannula can be replaced by a tube connected to the cannula, a catheter or a line.

Cannula fixation device 100 comprises a frame 120 composed of one or more segments and made of a rigid or semi rigid material. In the specific none limiting example illustrated in this drawing, cannula fixation device 100 has a symmetrical structure, therefore the right side and the left side of the device are similar in a mirror view and contain similar components. The symmetrical structure allows the medical team a convenient approach to all arteries and veins at the neck area of the patient without being limited to a specific side. however, nonsymmetrical structure having the major components illustrated herein at the right side or at the left side of the neck should also be construed as part of this invention and may be implemented as one optional embodiment of the invention.

In the specific example illustrated herein, frame 120 is configured to be mounted around the patient's neck and contains two main openings 122 at the right side of frame 120 and at the left side of frame 120 that serve as “windows” allowing the medical team a clear and convenient approach to the cannulation area. Frame 120 may be made of one or more segments. When it is made of more than one segment, the segments may be connected by one or more connectors 124. The usage of connectors may reduce the weight of the device and minimize the contact surface with the patient's body. Additionally, the connectors may be connected to frame segments 120 by common hinges, by clips, by strips and other connecting means. In some embodiments, connectors 124 may have a role in adjustment of the device to the specific physical dimensions of each patient and improvement of the fixation of the cannula. Additionally, the connectors 124 may provide certain flexibility to the rigid structure of frame 120.

In some optional embodiments, frame 120 may be designed to comprised areas with different dimensions and shapes relative to other areas of the frame as shown in the drawing. For example, the back side of frame 120 may be longer than the sides of the frame in order to provide support to the patient's neck.

In some alternative embodiments frame 120 or some of the frame segments have a partially closed structure. The dimensions of frame 120 may vary and the positioning of the device onto the patient's neck may vary accordingly. Thus, frame 120 may be in various height, thickness and width and may be positioned along the jaw line, near the shoulders and in between.

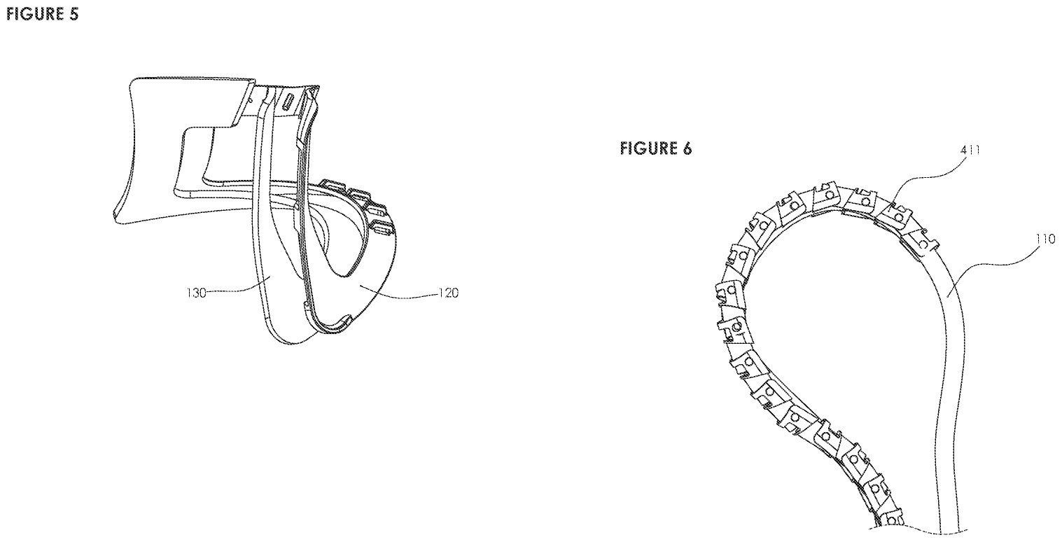

Yet, in further optional embodiments of the invention, the frame may have incomplete structure as illustrated in FIG. 5.

In accordance with some optional embodiments of the invention frame 120 is attached to an inner surface 130. Inner surface 130 (also denoted hereinafter: “inner layer”, “soft layer” and “inner soft layer” interchangeable) is preferably composed of a soft material and configured to provide the patient a pleasant and comfortable feeling at the contact area of the skin with device 100. Inner layer 130 may also be used to absorb fluid debris and body fluids secreted during the medical procedure at the cannulated area. Preferable but not necessarily, inner layer 130 is disposable. Inner layer 130 may overlap the entire surface of frame 120, or it may partially overlap frame 120, such that specific areas of frame 120 will not be covered at the inner side by soft layer 130. In some other areas of frame 120, inner layer 130 may extend beyond outer frame 120 to provide the patient soft support in specific areas if desired.

Optionally, cannula fixation device 100 comprise at least one holding element, configured to retain and secure cannula 110 to frame 120 along the axial extent of cannula 110. In the specific embodiment illustrated in FIGS. 1A-1B, a holding element designed as a series of projections 126 is shown. Although two projections 126 are sufficient for fixation of the cannula, it is preferred to have more than two (2) projections as it allows the medical team to divert cannula 110 and position it in a position that is not interrupting the medical procedure and interfere with the access to the treated area, as may occur when using only two projections 126 that dictate a specific, nonflexible retention point. Additional or alternative retention area may be located at the upper part of frame 120 adjacent to the cannulation area. At this location, the holding element may be an integral part of frame 120 or separated thereof. In the specific example illustrated herein, frame 120 comprises a curved shape creating a niche 128 and having two holes 1281 at the right and left sides of the niche that allows to secure cannula 110 to frame 120. Securing the cannula may be made by various securing elements available in the market such as but not limited to cable tie, clips, adhesive straps, thread, and the likes. Niche 128 may have a convex shape or a concave shape and cannula 110 may be positioned below frame 120 or above frame 120 according to the direction of the niche, respectively. In some optional embodiments of the invention frame 120 comprise only holes 1281 without having the curved niche 128. In such embodiments tube 110 is positioned between holes 1281 and secured to frame 120 by any of the securing elements mentioned above. It should be clear that additional affixing elements may be added and should all be construed as part of this invention. The affixing elements keep the incision location of the cannula on the patient's neck free of undesired obstructions and fixate the cannula safely.

After mounting cannula fixation device 100 on the patient's neck, the device should be fastened and the two edges of frame 120 should be connected one to the other in order to convert the device into its “ready to use” form. Fastening device 100 around the patient's neck and adjusting its diameter to the specific dimensions of the patient and connecting the two edges of frame 120 may be made by various securing elements available in the market. In the specific, none limiting example illustrated in these figures, closing device 100 into a functional form is performed by using a Velcro strap 140 that is easy to use and allows to tighten device 100 into the desired dimensions of the patient. The Velcro strap 140 may be fixed to frame 120 by dedicated hinge 141 at one side of frame 120 and inserted through a dedicated slot 142 at the other side of frame 120 to thereby allow closing and fastening of device 100 to its functional form. Although in the specific example illustrated in these figs a single Velcro strap is used, it should be clear that additional straps may be implemented on a similar concept. Optionally, cannula fixation device 100 may further include a chin support 150. Chin support 150 is preferably positioned below the lower jawbone and configured to support the patient's head from the front and minimize undesired movements. Chin support 150 may also function as an additional fastening element and replace at least one of the Velcro straps 140 as illustrated in FIG. 1A.

One skilled in the art will readily recognize multiple alternate detailed designs, construction materials, and fixed/adjustable sizes in which frame 120 and the additional elements connected therethrough may be realized for adapting to multiple different applications, neck sizes and for providing improved comfort level to patients. For example, frame 120 may be constructed with a semi-rigid material, inner layer 130 may be constructed with a soft, fast drying material etc. Furthermore, additional attachment means for supporting/affixing further medical cannulas and other medical equipment may be included.

Further shown in FIGS. 1A-1B, coil 111 that is arranged around cannula 110, at the section of the cannula between the two holding areas: niche 128 and projections 126. Coil 111 is an optional accessory configured to enable a limited sliding displacement of cannula 110 within this section of the cannula, for preventing undesirable axial loading (i.e., pull) or collapse/kink of cannula 110 that may occur for example, when a patient bends his head towards one of his/her shoulders, or upon conversion of the patient positioning during proning. The support provided by coil 111 allows fluent blood flow or other fluids flow through the cannula. Supportive element 111 may have a different design as illustrated in FIG. 5 hereinbelow. Optionally, coil 111 or portions thereof are made of a rigid or semi-rigid material, for enabling a desirably adjusted curvature of the cannula or a guide tube. Other supportive elements for cannula 110 may also be used and should all be construed as part of this invention.

Also shown in these drawings are the cannula proximal tip 1101 the is tightly adjacent to the patient's body at the cannulation area, and the tube distal tip 1102 that is further connected to a machine such as ECMO, cardiopulmonary bypass machine, dialysis machine, and plasmapheresis machine.

FIGS. 1C and 1D are schematic isometric illustration of cannula fixation device 100 of FIG. 1A in an open form ready to be mounted on a patient 70, and the device mounted on a patient's neck respectively. As shown in these drawings, in an open form, chin support 150 and Velcro strap 140 are both released and allow to easily mount device 100 around the patient's neck. Upon mounting the device, both the Velcro strap 140 and the chin support 150 are closed and tightened according to the patient's physical dimensions and anatomical structure. Also shown in these drawings are openings 122, projections 126, cannula 110, niche 128, holes 1281, slot 142, tube proximal tip 1101 that is tightly adjacent to the patient's body at the cannulation area, and the cannula distal tip 1102 that may be connected to any relevant medical machine.

FIG. 1E is a schematic front view illustration of cannula fixation device 100 of FIG. 1A mounted on a patient 70 in a close ready to use form. In such position, the neck blood vessels 72 of the patient are passing through the large openings 122R and 122L of frame 120 and allow a convenient bi-lateral approach to the neck vessels 72 that enables performance of cannulation and other medical procedures needed at the neck area, while securing and fixating the cannula inserted.

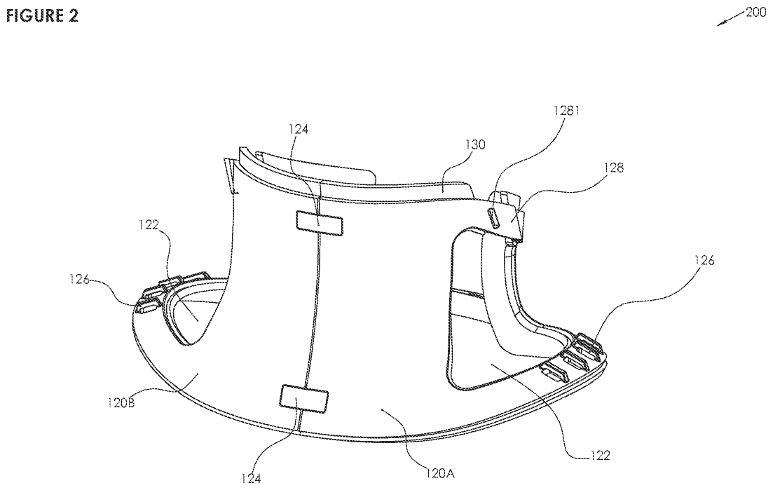

FIG. 2 is a schematic illustration of a cannula fixation device 200 according to one another embodiment of the invention. In this specific embodiment, frame 120 is composed of two main sections 120A and 120B connected to each other by two connectors 124, said connectors are positioned at the upper and lower back side of frame segments 120A and 120B. In the specific embodiment illustrated herein the back side of the entire frame is made of a massive structure that covers the entire back side of the patient's neck and support it, with a limited mobility and flexibility at the back side of cannula fixation device 200, however the sides of the device comprise large right and left openings 122 that allows clear and convenient approach to the neck vessels of the patient and cannulation area. Other features of device 200 are similar to the features described hereinabove with reference to cannula fixation device 100. Also shown in this drawing are projections 126, inner soft layer 130, niche 128 and holes 1281 for securing the tube to the frame around niche 128.

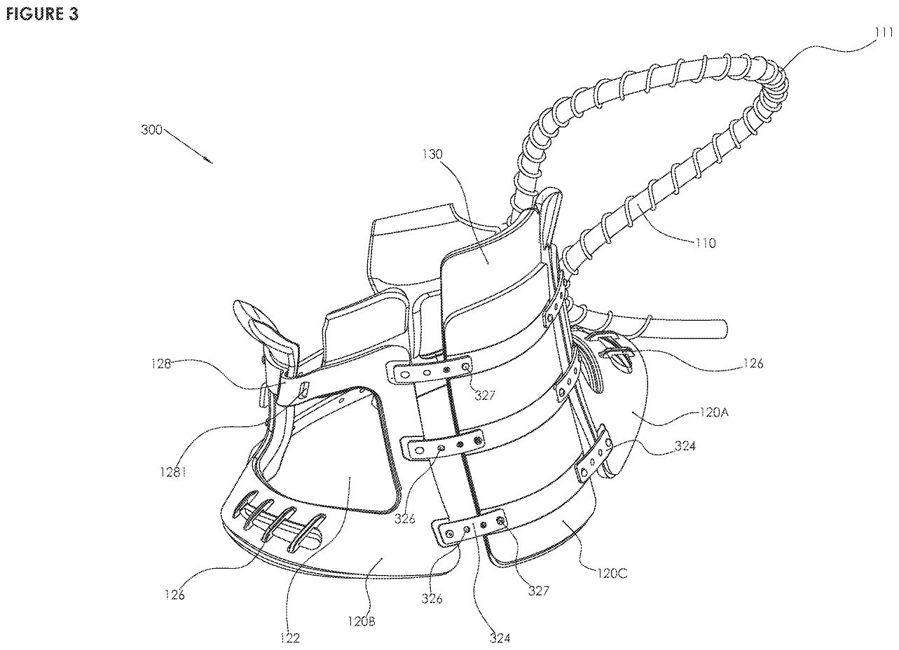

FIG. 3 is a schematic illustration of a cannula fixation device 300 according to one another embodiment of the invention, also designed as a cervical collar arranged around the patient's neck and extend between the patient's shoulders and lower jaw. In the specific example illustrated in this drawing, device 300 comprises three (3) frame segments, one at the rear for neck support 120C, and two symmetrical frames 120A and 120B that are used as side supports, while all frame segments are connected one to the other by one or more connecting straps 324 therebetween. In some optional embodiment, frame segments 120A, 120B and 120C may have fixed heights. Alternatively, they may be adjustable in height, for accommodation of patients with different neck sizes.

Connecting traps 324 are preferably made of flexible materials that enable limited stretching, and correspondingly, spacing between middle frame 120C and side frames 120A and 120B for enabling an effortless and comfortable wearing of cannula fixation device 300 on a patient. In this example, connecting straps 324 comprise a set of holes 326 suitable to be reversibly fixed on pins 327 attached to frame 120 at the lateral sides of the frame, in a manner that the medical team adjust the specific hole 326 to be instead to the frame's pin 327 according to the dimensions of the specific patient, thereby adjusting the dimensions of device 300 to the specific patient wearing the device and tightening it to the patient's neck to get optimal fixation of the cannula.

Generally, side frames 120A and 120B at the bottom side are configured to rest on the patient's shoulders. Projections 126 in this embodiment are in different sized to allow strong and effective fixation of different diameter cannulas. Inner layer 130 is extended beyond outer frame 120 to provide maximal comfort to the patient.

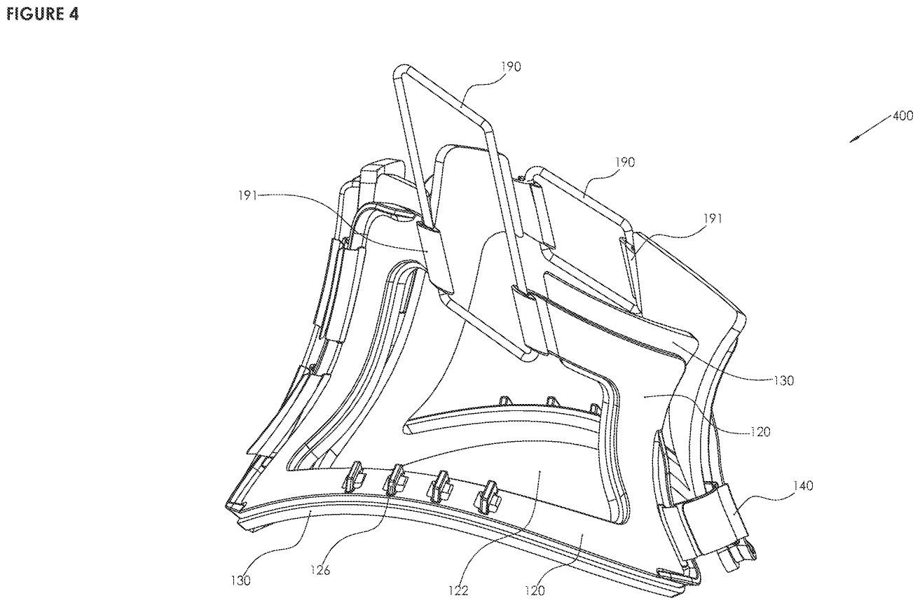

FIG. 4 is a schematic illustration of a cannula fixation device 400 according to one another embodiment of the invention. In this specific embodiment the upper portion of frame 120 is incomplete and the two edges of frame 120 at the open area comprise hinges 191 that holds a mini frame 190. Mini frame 190 provides a space to make incision through it and allows to pass a cannula inserted at the jaw area. Additionally, it may serve as additional holding element that supports to fixate the cannula. Also shown in this drawing are inner layer 130, projections 126 and openings 122. The incomplete structure of the frame 120 may be on one side or on both sides as illustrated in the example provided herein. Additionally, although the gap is at the upper bar of the frame it may also be located at the lower bar of the frame on the same concept. Similarly, the gap is not necessarily comprising a mini frame that connect the two edges of the frame and other supportive elements or no elements at all may be used between the open two edges of the frame.

FIG. 5 is a schematic partial close-up view demonstrating the two layers of cannula fixation device 100, the outer layer denoted above frame 120 and inner soft layer 130 separated thereof for clarity purposes. As mentioned in the above, both layers may be reversibly attached to each other of fixedly attached. Both layers may be disposable or non-disposable, or only one of them may be made of disposable material. The inner soft layer 130 may partially or fully cover frame 120. In some optional embodiments it may extend beyond frame 120 to further provide soft and pleasant support to the patient and soften the stiffness feeling that rigid frame 120 may cause. Soft layer 130 may further have a role in absorbing fluids at the neck area during cannulation. It should be clear to a man skilled in the art that inner layer 130 is only optional and in some implementations of this invention it may be omitted.

FIG. 6 is a close-up view of a cannula 110 partially wrapped by a supporting accessory 411 for preventing its collapse/kinking according to some embodiments of the invention. Supporting accessory 411 has similar role as supporting accessory 111 shown in FIG. 1A and is configured to be mounted on a cannula/tube upon usage of the cannula fixation device of the invention at least at the cannula section that is being retained by the fixation device. In the specific example illustrated in this drawing, supporting accessory 411 is composed of set of rings that are mounted onto the cannula and prevent its collapse. The rings may be connected to each other or separated thereof, and they may be made of complete circle or partially open rings. Other designs for the same purpose should all be construed as being part of this invention.

The described cannula fixation device, embodiments of which are described hereinabove, is highly useful for affixing various medical cannulas, tubes, catheter and lines and in particular during blood treatment operations through a central vein such as the internal jugular vein. The cannula fixation device provides a secured fixation, thereby preventing undesirable pulling or displacement of the cannula, even over a long period of use and in any body position of the patient including proning.

Although embodiments of the invention have been described by way of illustration, it will be understood that the invention may be carried out with many variations, modifications, and adaptations, without exceeding the scope of the claims.

1. A cannula fixation device comprising at least:
a. an open frame configured to be mounted on a patient's neck, said frame is encircling the patient's neck and having at least one opening at the cannula entry point for allowing approach to at least one neck artery and/or vein;
b. at least one cannula holder configured to allow securing of a cannula to said frame; and
c. at least one fastening element for closing the open frame onto the patient's neck and adjusting it to the neck's dimensions;
wherein said cannula fixation device is configured to secure at least one cannula inserted into the neck artery and/or vein of said patient and preserve the cannula position within the artery and/or vein in any body positioning, while said patient is lying down, standing, or sitting.

2. A cannula fixation device according to claim 1, wherein said frame is made of a rigid or semi-rigid material.

3. A cannula fixation device according to claim 1, wherein said frame is composed of two or more frame sections sequentially connected to each other.

4. A cannula fixation device according to claim 3 further comprising connectors for connecting said frame sections.

5. A cannula fixation device according to claim 1, wherein said frame comprises a left opening and a right opening so as to enable simultaneous approach to the left and right neck arteries and veins.

6. A cannula fixation device according to claim 1, further comprising an inner soft layer attached to said frame and covers said frame either fully or partially.

7. A cannula fixation device according to claim 1, wherein said at least one cannula holder is a part of said frame or an independent connecting element attached thereto.

8. A cannula fixation device according to claim 1, wherein said at least one cannula holder is a curved section of said frame creating a niche.

9. A cannula fixation device according to claim 8, wherein said niche is having two holes at the right and left sides of the niche that allows to secure the cannula to the frame.

10. A cannula fixation device according to claim 1, wherein said at least one cannula holder is a selected from the group consisting of: clips, straps, adhesive tapes, at least two subsequent mechanical projections, clasp, cable tie, and plastic or nylon restraint.

11. A cannula fixation device according to claim 1, wherein said frame further comprises at least two projections configured to hold the ascending portion the cannula and fixate it to the frame.

12. A cannula fixation device according to claim 1, wherein said frame is composed of different heights at different areas around the neck.

13. A cannula fixation device according to claim 1, wherein said at least one fastening device is selected from the group consisting of: Velcro strap, elastic modular strap, clips, straps, adhesive tape, and clasp.

14. A cannula fixation device according to claim 1, wherein said patient is intubated for either one of the following: extracorporeal blood processing, cardio-vascular therapeutic or diagnostic procedure, hemodialysis, plasmapheresis, transvenous cardiac pacing, invasive hemodynamic monitoring, and heart-lung machine.

15. A cannula fixation device according to claim 1, wherein said patient is positioned in a proning position.

16. A cannula fixation device according to claim 1, wherein said secured cannula is further connected to a tube, and said tube is functionally connected to the frame by said cannula holders.

17. A cannula fixation device according to claim 16, wherein said tube is supported by a rigid assembly mounted on the portion of the tube that is attached to the frame so as to prevent kink/collapse of said tube.

18. A cannula fixation device according to claim 17, wherein said rigid assembly is either one of the following: a metal/plastic coil, rigid rings, u shapes, and hooks.

19. A cannula fixation device for securing at least one cannula inserted into the neck artery and/or vein of a patient and preserve the cannula position within the artery and/or vein in any body positioning, said cannula fixation device comprising at least:
a. an open frame comprising one or more frame segments and configured to be mounted on a patient's neck, said frame having at least one opening in dimensions allowing to approach at least one neck artery and/or vein at the cannula entry point for performing a medical procedure;
b. at least one cannula holder configured to allow securing and/or fixating of a cannula inserted into the patient's body to said frame; and
c. at least one fastening element for closing the open frame onto the patient's neck to encircle the neck and adjusting it to the neck's dimensions.
